(19) United States
(12) Patent Application Publication
(10) Pub. No.: US 2011/0120473 A1
(43) Pub. Date: May 26, 2011

(54) Regulation of intrathoracic pressures by cross seal vent valve

(75) Inventor: Samuel David Piper, Carmichael, CA (US)

(73) Assignee: Piper Medical, Inc., Carmichael, CA (US)

(21) Appl. No.: 12/927,755

(22) Filed: Nov. 22, 2010

Related U.S. Application Data
(60) Provisional application No. 61/283,023, filed on Nov. 24, 2009.

(51) Int. Cl. A61M 16/20

(52) U.S. Cl. 128/207.16

(57) ABSTRACT

The present invention relates generally to devices and methods for finite control and regulation of patient intrathoracic pressures, and more specifically, to devices and methods that finitely regulates a patient's intrathoracic pressures during repeated cycling events (i.e. respiration) by use of a cross-seal vent valve to form transient pressure windows. The cross-seal vent valve is biased against the pressure necessary to evacuate and/or inflate the lungs of that patient, while a controlled venting of that pressure by at least a partial volume thereof allows for controlled resetting of the baseline pressure to anatomical norms. This enhanced means for regulating intrathoracic pressure are applicable in a number of medically important therapies, including but not limited to, conditioning of pulmonary systems for acclimation to altered environmental conditions, reconditioning of pulmonary system after operating in a diminished state, and application in cardiopulmonary resuscitation procedures.

### CROSS-REFERENCE TO RELATED APPLICATIONS

This application claims the benefit under 35 U.S.C. 119(e) of U.S. provisional application Ser. No. 61/283,023 filed Nov. 24, 2009, which is incorporated by reference herein in its entirety

### STATEMENT REGARDING FEDERALLY SPONSORED RESEARCH

Not Applicable

### BACKGROUND OF THE INVENTION

The present invention relates generally to devices and methods for finite control and regulation of patient intrathoracic pressures, and more specifically, to devices and methods that finitely regulates a patient's intrathoracic pressures during repeated cycling events (i.e. natural or artificial respiration) by use of a cross-seal vent valve. The enhanced means for regulating intrathoracic pressure during patient respiration are applicable in a number of medically important therapies, including but not limited to, conditioning of pulmonary systems for acclimation to altered environmental conditions, reconditioning of pulmonary system after operating in a diminished state, and application in cardiopulmonary resuscitation procedures.

Intrathoracic pressure as related to a patient is a measure of performance generated in the thoracic region defined in the upper chest as the volume between the posterior T10 spinous process, the anterior xiphoid process of the ribcage and bounded distally by the diaphragm (Clemente, C. D. (1981). Anatomy). The lungs of the patient are encircled by the thoracic region. As autonomous or artificial breathing occurs, during inspiration the diaphragm descends, the ribcage elevates, and the intrathoracic region volumes increases thereby creating a decreased intrathoracic pressure. Gas exterior to the patient (either environmental or artificially introduced) is drawn into the lungs as a result of this decreased or negative pressure. Upon expiration, the diaphragm ascends; the ribcage descends causing a decrease in intrathoracic volume and an increase in intrathoracic pressure. Gas within the patient's lung is then at a higher pressure than the environment, and thus the gas is expelled from the lung.

Under normal circumstances (i.e. a patient with a healthy respiratory system) the cycling of positive and negative pressures within the intrathoracic region occurs in a continuous, regular pattern as a result of normal respiration. However, this pattern can be interrupted in terms of frequency and/or amplitude due to such effectors as induced respiratory stress (e.g. exercise), diminished respiratory capacity (e.g. disease or injury) and compromised respiratory performance (e.g. cardiopulmonary collapse). While the first effecter, induced respiratory stress, is focused on an incremental improvement in respiratory performance, the latter two effectors are emergent in nature and require medical attention in order to sustain sufficient respiration and maintain patient viability. To counter each of these effectors, it is desirable to introduce a means into the patient respiratory tract such that the degree and duration of positive and/or negative pressures attained during respiration cycling is finitely regulated.

An incremental improvement in respiratory performance is desirable by individuals who require an enhanced ability to cycle oxygen into their systems. Athletes who subject their systems to sudden or prolonged stress can benefit from artificially restricted or altered respiratory environments. Use of an artificially restricted respiratory environment causes the individual's intrathoracic region to develop with higher capacities, greater musculature, and quicker recovery times. Therefore, when an athlete trains in an artificially restricted respiratory environment, and that environment is then removed, the individual then enjoys a higher respiratory capacity (“Update in the understanding of respiratory limitations to exercise performance in fit, active adults”, Dempsey, et al., Chest 2008, September; 134(3), incorporated by reference herein its entirety). Similarly individuals who must operate in environments where atmospheric pressure is at extremes, such as deep sea diving and high altitude climbing, may also benefit by enhanced respiratory development and capacities. Such incremental improvements can also be desirable to individuals who suffer from chronic pulmonary or respiratory condition or are recovering from a reduced pulmonary or respiratory condition or injury.

Diminished respiratory capacity is evident in patients, which have either existing damage or disease in the lung or other elements of the respiratory, pulmonary, and circulatory tracts or injury caused to an element of the system by accident or surgical intervention. In the situation wherein the respiratory system is damaged, it can be desirable to integrate into a gas supply a finite ability to control the positive and negative pressures created in the intrathoracic region so as to minimize further damage, improve performance, and assist in reconditioning of the system. Numerous modes of injury and damage to the respiratory system exists, as generally taught in the following published citations, incorporated by reference herein in their respective entireties: “Clinical review: Positive end-expiratory pressure and cardiac output”, Luecke, et al., Critical Care. 2005; 9(6); “Physiological changes occurring with positive pressure ventilation”, Robb, Intensive Critical Care Nurse 1997 October; 13(5); and, “Is there a best way to set positive expiratory-end pressure for mechanical ventilatory support in acute lung injury?”, MacIntyre, Clinical Chest Med. 2008 June; 29(2).

Compromised respiratory performance is defined as failure of the respiratory system to cycle, often as an element of complete cardiopulmonary collapse. Cardiopulmonary collapse, or sudden cardiac arrest, is a major cause of death worldwide and is the result of a variety of circumstances, including heart disease and significant trauma. In the event of a cardiac arrest, a rapid and appropriate response is essential in order to improve a patient's chance of survival by at least partially restoring the patient's respiration and blood circulation. External chest compression technique generally referred to as cardiopulmonary resuscitation (CPR) is the most common means of attaining partial respiration and circulation in a patient.

Intrathoracic pressure is momentarily increased through application of external force as part of the CPR procedure. An increase in intrathoracic pressure induces blood movement from the region of the heart and lungs towards the peripheral arteries. Such pressure increase partially restores the patient's circulation. Traditional CPR is performed by actively compressing the chest by direct application of an external pressure to the chest. After active compression, the chest is allowed to expand by its natural elasticity which causes expansion of the patient's chest wall. This expansion allows some blood to reenter the cardiac chambers of the heart. The procedure as described, however, is insufficient to induce sufficient respiration in the patient. To attain respiration, conventional CPR also requires periodic ventilation of the patient. This is commonly accomplished by mouth-to-mouth technique or by using positive-pressure devices, such as a self-inflating bag, which relies on squeezing an elastic bag to deliver gas into the patient's respiratory system.

With CPR, and other similar techniques, an increase in the amount of venous blood flowing into the heart and lungs from the peripheral venous vasculature is desirable to increase the volume of oxygenated blood leaving the thorax during the subsequent compression phase. It would therefore be desirable to provide improved methods and apparatus for enhancing venous blood flow into the heart and lungs of a patient from the peripheral venous vasculature as well as enhancing blood leaving the thorax during CPR. Further, it would be particularly desirable to provide techniques which would enhance oxygenation and increase the total blood return to the chest during the decompression step of CPR and increase the total blood flow leaving the thorax during the compression set of CPR.

Improvement in oxygenation and blood flow can be accomplished by regulating intrathoracic pressure, thereby amplifying the total intrathoracic pressure swing. U.S. Pat. Nos. 6,986,349; 6,604,523; 6,526,973; and 6,425,393 to Lurie et al., each incorporated by reference it their respective entireties, teach to use of a valve type impingement in the respiratory tract of a patient utilizing continuous vacuum application. Upon analysis of such a device as described by Lurie, et al., it is found that by continuous application of a set vacuum, an initial enhancement is obtained in a first CPR compression cycle, but the benefit is diminished over subsequent cycles as the set vacuum effectively establishes and maintains the intrathoracic cavity to a finite lower pressure point.

There exists a need for a means to finitely regulate the intrathoracic pressure of a patient, which is readily applied to a patient and offers improved means for controlling the positive and/or negative pressures achieved in the intrathoracic region, the rates by which those pressures are developed and allows for controlled resetting of the baseline pressure to anatomical norms.

### SUMMARY OF THE INVENTION

The present invention relates generally to devices and methods for finite control and regulation of patient intrathoracic pressures, and more specifically, to devices and methods that finitely regulates a patient's intrathoracic pressures during repeated cycling events (i.e. respiration) by use of a cross-seal vent valve. The cross-seal vent valve includes a piston biased against the pressure necessary to evacuate and/or inflate the lungs of that patient, while a controlled venting of that pressure by at least a partial volume thereof allows for controlled resetting of the baseline pressure to anatomical norms. The valve requires a threshold pressure be exceeded, which in turn opens the valve, and the valve remains in an open position so long as the threshold is maintained or exceeded. Typically the threshold pressure is defined by the amount of force required to overcome a contra-acting force provided by a biasing means, such as a helical-coil spring. The enhanced means further includes a cross-seal vent allowing for continuous venting of pressure necessary to inflate or exhaust the lungs of that patient by at least a partial volume thereof. Use of the cross-seal vent enables the cross-seal vent valve assembly to return the intrathoracic cavity to anatomical norms during each subsequent inhalation/exhalation sub-cycle. The enhanced means for regulating intrathoracic pressure are applicable in a number of medically important therapies, including but not limited to, conditioning of pulmonary systems for acclimation to altered environmental conditions, reconditioning of pulmonary system after operating in a diminished state, and application in cardiopulmonary resuscitation procedures.

In a first embodiment, a patient connector is in fluid communication with a cross-seal vent valve assembly, wherein the valve assembly comprises a valve piston associated with and biased against an exhalation port of the assembly and a check valve is associated within the valve piston which allows for controlled gas release. The valve piston base engages upon an interior aspect of a valve assembly cap. The valve piston is maintained against an interior aspect of a valve assembly cap by the force exerted by a biasing means (e.g. helically wound spring), though it is within the purview of the present invention that the biasing means can be mechanically or electronically adjusted through manual, semi-automated and fully automated processes. The biasing means is retained in the assembly by a piston guide, which itself includes a plurality of atmospheric vents. As pressure from a chest compression (or induced tidal volume) through a patient connector through the valve base and against the valve piston base, exhalation gas is dispersed through the check valve. A vacuum is then developed within the valve assembly as a result of elastic expansion of the intrathoracic chamber which is held in check by the biasing means. The biasing means maintains the valve piston closed until an initial threshold pressure is exceeded. Once the pressure exceeds the force exerted by biasing means, the valve piston will translate from a closed to an open condition, and will remain open until sufficient pressure is dissipated as to allow biasing means to return the valve piston base to a closed position against the interior aspect of the valve assembly cap. The rate at which the pressure drops from the opening pressure to the closing pressure is regulated in part by the restrictive value of cross-seal vent associated with the assembly. Conversely, the cross-seal vent allows for continuous pressure regulation in both exhalation (increased pressure) and inhalation (reduced pressure), which in combination with the valve piston, allows for variable pressure rates during cycling and, importantly, allows the valve assembly to reset to anatomical norms for improved cardiac flow.

It is critical to note that the operation of the aforementioned valve assembly allows for an initial higher pressure to cause the piston to open and to then subsequently allow a second lower pressure being achieved due to equalization flow through the cross-seal vent. By setting the threshold pressure and the restriction to flow of the cross-seal vent patient parametrics, including lung capacity, the pressure within the intrathoracic region can be specifically regulated. Those skilled in the art can appreciate that a number of alternate piston and biasing schemes could be employed without departing from the nature of the invention. By impeding inhalation gas flow, the present invention specifically regulates the application and retention of vacuum pressure within the thorax, which is briefly maintained and then relinquished in a controlled and regulated manner, eventually allowing the pressure in the thorax to return to an ambient baseline state due to the equalization flow through the cross-seal vent. This is superior to normal CPR techniques without the invention as in such a case the CPR compression would primarily be functioning to simply push gas in and out of the patient's lungs and thus would result in far less pressure variance in the thorax, resulting in far less blood flow through the thorax. Furthermore, the invention has the further advantage of providing feedback to the clinician or operator on whether sufficient chest compression has been supplied (as indicated by movement of the piston).

Although a constant vacuum only methodology has been shown to be useful, the current invention is more effective based on a theory that substantially all the additional blood flow into the heart during a finite vacuum phase occurs initial chest compression, and without resetting the thorax back to a nominal lower pressure prior to subsequent chest compressions little or no additional blood flow is realized above that which is produced during un-assisted CPR. In contrast to a constant vacuum methodology, where there is no added benefit of holding vacuum throughout the entire decompression phase of CPR, and doing so impedes the success of subsequent chest compressions. The current invention provides suitable magnitude and duration of vacuum necessary to realize the added blood flow of the initial chest compression of a constant vacuum methodology, and then beneficially allows the thoracic cavity to return to ambient pressure (prior to the subsequent chest compression), setting the stage for equally effective subsequent chest compressions. To present this point in alternate wording: a constant vacuum methodology is primarily effective on the first chest compression and not on subsequent chest compressions, until such time that a breath is delivered to the patient and at which point the thoracic cavity is effectively reset to ambient pressure by this manual and deliberate action. The current invention is as effective on the first chest compression, and equally effective on every subsequent chest compression thereafter since it automatically resets the baseline thoracic cavity pressure back to ambient pressure after the enhanced pulmonary blood flow has been realized but before the subsequent chest compression. The current invention requires no manual resetting to ambient pressure, and is therefore more suited to CPR situations in which there is only one rescuer, when the CPR standards direct focus onto chest compressions and not breaths.

The above embodiments can be used with a sealing mask, endotracheal tube, or any other equivalent respiratory patient engagement or sealing means.

According to the present invention, use of a finitely regulated cross-seal vent valve assembly can be readily employed for increasing cardiopulmonary circulation induced by chest compression and decompression when performing cardiopulmonary resuscitation. Particularly advantageous is in the use of a cross-seal vent valve assembly biased against patient inhalation, and any one of the described (equivalent/alternate) control means biased against patient inhalation, is that a “transient pressure vacuum window” can be created in the cycling of the negative pressures formed in the intrathoracic region of a patient during CPR. This “transient pressure, vacuum window” is a point where, due to biasing of the respective control means of a cross-seal vent valve assembly attached to the respiratory system of a patient, regulated inhalation can occur within a finite set of conditions and therefore a set negative pressure is retained within the intrathoracic region for a finite period of time thus enhancing cardio-pulmonary flow. The methods and devices may be used in connection with any generally accepted CPR methods or with active compression-decompression (ACD) CPR techniques. When a valve assembly in accordance with the present invention is used with CPR methods, the “transient pressure vacuum window” automatically coincide with steps in the CPR method such that less force is lost in movement of air volumes from the patient's thoracic region and incremental pressure gains are achieved in inducing circulation of oxygenated blood in the patient (measured as blood flow).

Cardiopulmonary circulation is increased according to the invention by impeding air flow into and/or out of a patient's lungs during the compression and/or decompression phase. This increases the magnitude and prolongs the duration of positive and/or negative intrathoracic pressure during compression and the subsequent decompression of the patient's chest and result in increases of venous blood flow into the heart and lungs from the peripheral venous vasculature during decompression and also results in increases in oxygenated blood leaving the thorax during compression. Thus the present invention results in the greater inflow and outflow of blood through the heart and lung corresponding with the initiation of compression and decompression accompanying CPR rather than the diminished blood flow and the increased flow of gases coming in and out of the lung that would result without the invention. As the inventive concept provides for a return to a baseline lung pressure prior to the each subsequent chest compression, the invention has the further advantage over other technologies of still allowing gas exchange as a result of CPR and works harmoniously with various ventilation technologies and artificial breathing techniques.

In a specific embodiment, impeding the air flow into the patient's lungs is accomplished by altering ventilation during the decompression phase of CPR through use of a cross-seal vent valve assembly having the ability to finitely regulate the intrathoracic pressure of the patient. The piston valve is biased to open and permit the inflow of an increased air volume when the intrathoracic pressure falls below a threshold level. The invention further allows for periodically ventilating the patient by allowing the provision of positive pressure gas into the gas inlet/ambient port of the cross-seal vent valve assembly whether by use of a ventilator technology, manual resuscitator, or other artificial breathing techniques.

When performing cardiopulmonary resuscitation to enhance circulation according to the invention, an operator compresses a patient's chest to force blood out of the patient's thorax. The patient's chest is then decompressed to induce venous blood to flow into the heart and lungs from the peripheral venous vasculature either by actively lifting the chest (via ACD-CPR) or by permitting the chest to expand due to its own elasticity (via conventional CPR). During the decompression step, air flow is impeded from entering into the patient's lungs which enhances negative intrathoracic pressure and increases the time during which the thorax is at a lower pressure than the peripheral venous vasculature. Thus, venous blood flow into the heart and lungs from the peripheral venous vasculature is enhanced during decompression as a result of enhanced venous return rather than from inflow of air via the trachea. In a particular embodiment, compression and decompression of the patient's chest may be accomplished by pressing an applicator body against the patient's chest to compress the chest, and lifting the applicator to actively expand the patient's chest.

Any of the above embodiments may further include one or more CPR assistant devices into the cross-seal vent valve piston assembly, wherein one or more visual and/or aural signals are provided to the operator of the device for effectively conducting CPR (i.e. pace or rate, measure of applied force) and/or patient condition (i.e. pulse, return of autonomic function/respiratory response).

Other features and advantages of the present invention will become readily apparent from the following detailed description, the accompanying drawings, and the appended claims.

### BRIEF DESCRIPTION OF THE DRAWINGS

The invention will be more easily understood by a detailed explanation of the invention including drawings. Accordingly, drawings which are particularly suited for explaining the inventions are attached herewith; however, it should be understood that such drawings are for descriptive purposes only and as thus are not necessarily to scale beyond the measurements provided. The drawings are briefly described as follows:

FIG. 1 is a perspective view of a valve assembly in accordance with an embodiment of the present invention;

FIG. 2 is an exploded perspective view of a valve assembly in accordance with FIG. 1;

FIG. 3 is an additional exploded perspective view of a valve assembly in accordance with FIG. 1;

FIG. 4 is a front side view of a valve assembly in accordance with FIG. 1, it should be noted that the back side, left side, and right side views are equivalent to the front side view;

FIG. 5 is a cross-sectional view taken along line shown in FIG. 4;

FIG. 6 is a top side view of a valve assembly in accordance with FIG. 1; and

FIG. 7 is a bottom side view of a valve assembly in accordance with FIG. 1.

### LIST OF REFERENCE NUMERALS

2 Patient Connector Port
4 Check Valve Flapper
6 Check Valve Retention
8 Valve Piston
10 Check Valve Flow Conduits
12 Biasing Means
14 Valve Base
16 Valve Top
18 Piston Guide
20 Cross-Seal Vent
22 Ambient/Ventilation Port
24 Piston Shaft
26 Central Piston Conduit
28 Retention Boss
30 Flapper Orifice
50 Cross-Seal Vent Valve Assembly

### DETAILED DESCRIPTION OF SPECIFIC EMBODIMENTS

While the present invention is susceptible of embodiment in various forms, there is shown in the drawings and will hereinafter be described a presently preferred embodiment of the invention, with the understanding that the present disclosure is to be considered as an exemplification of the invention, and is not intended to limit the invention to the specific embodiment illustrated. Herein, the term “exhalation” is used as to describe any event, whether voluntary on the part of the patient or not, which results in any amount of expelled gas or pressure from the patient. Similarly, the term “inhalation” is used to describe any event that results in any receipt of gas by the patient, or a vacuum pressure relative to ambient air.

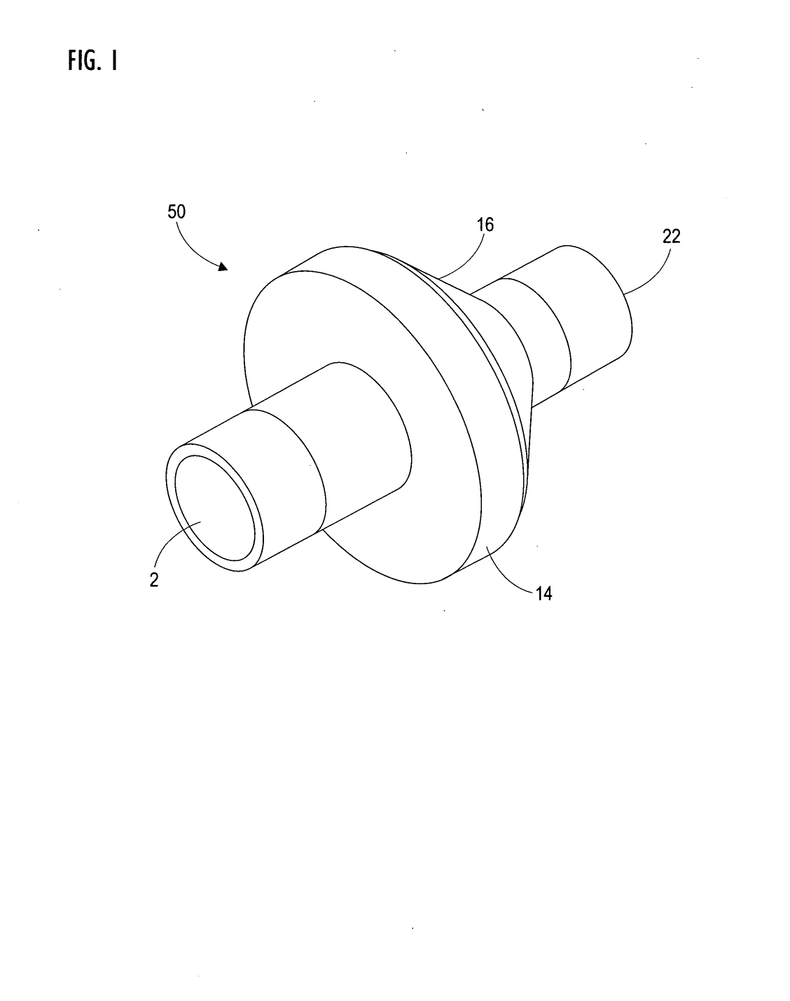

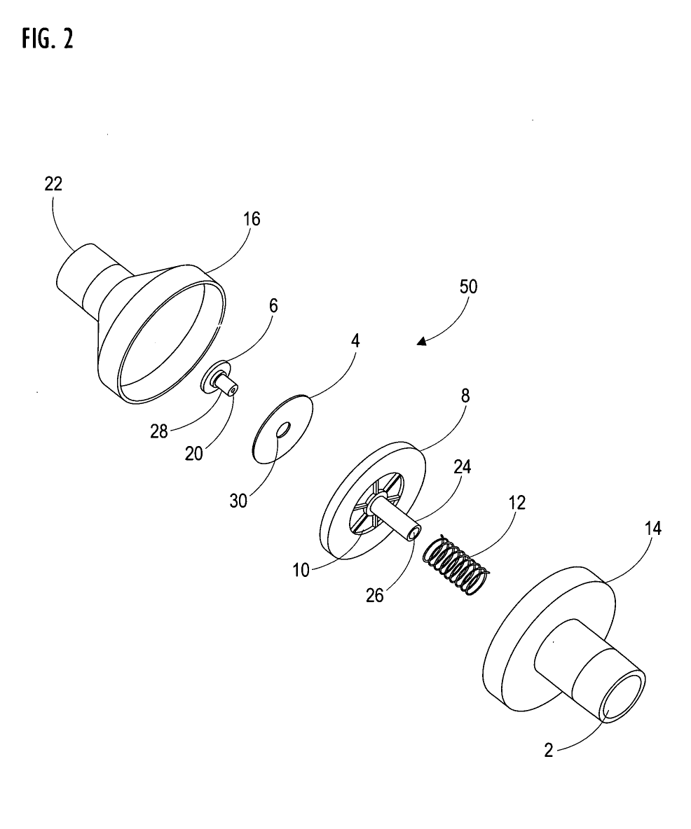

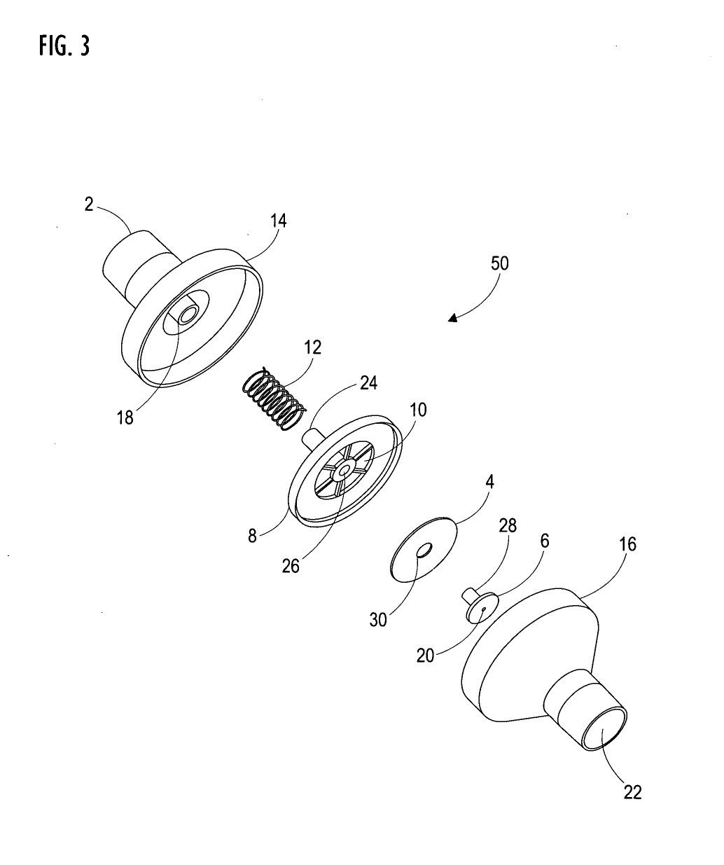

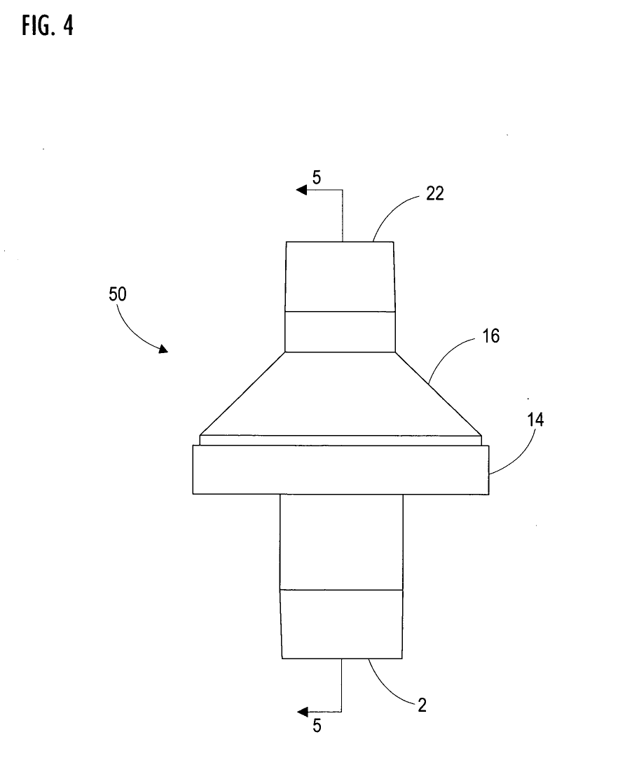

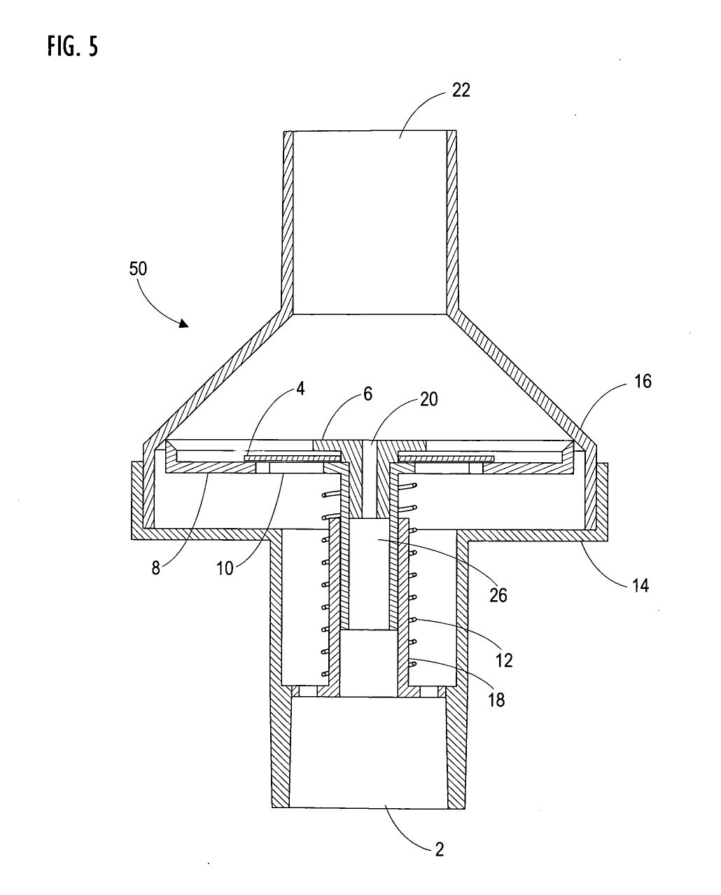

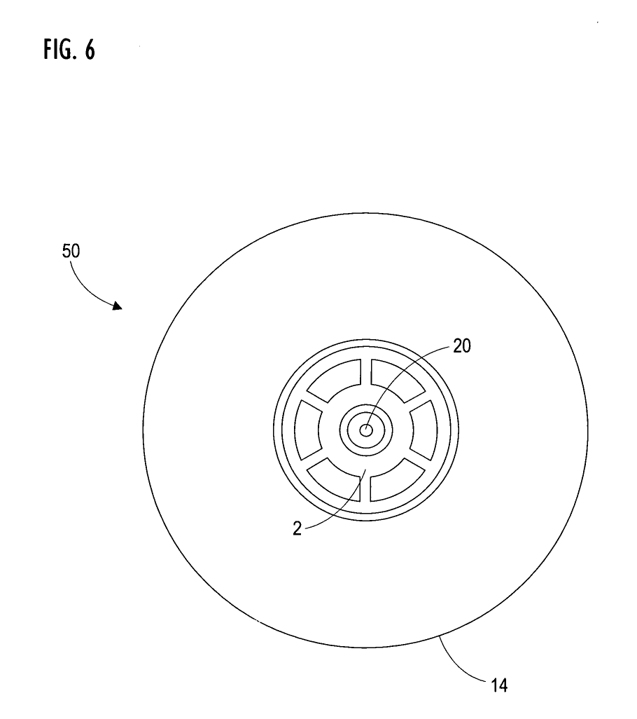

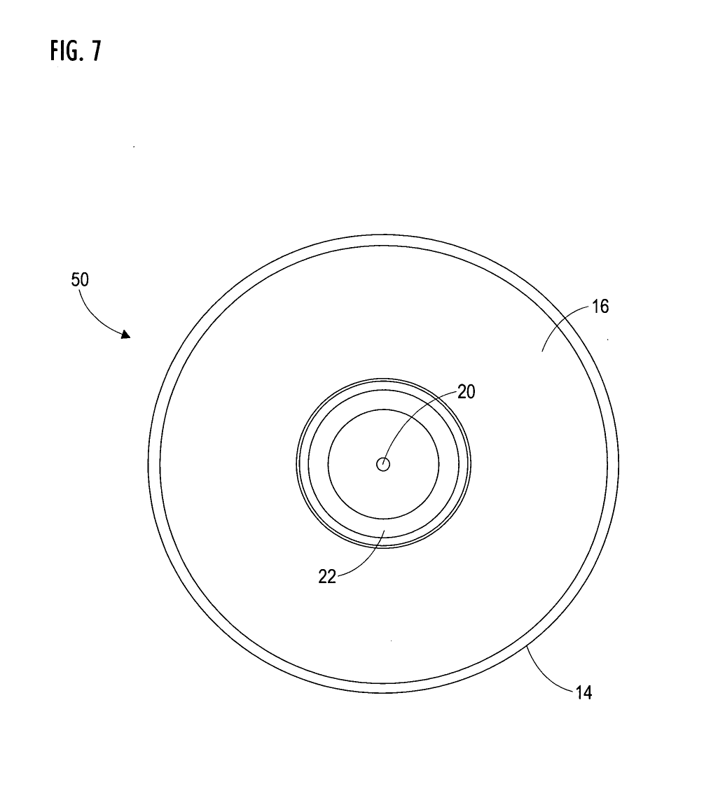

FIG. 1 through 7 depicts a first embodiment of the present invention wherein the primary goal of achieving a valve assembly with enhanced intrathoracic pressure regulation is achieved. A cross-seal vent valve assembly 50 comprises a patient connector port 2 in fluid communication with a valve piston 8 enclosed by a valve top 16 and valve base 14, wherein valve piston 8 defines a diameter and surface area of exposure upon valve top 16. Copending from the valve piston 8 is piston shaft 24. Piston shaft 24 extends through and is retained by piston guide 18 such that piston guide 18 allows piston shaft 24 and thereby valve piston 8 to move in a linear relationship relative to valve base 14 and valve top 16. In the embodiment depicted in FIG. 1 through 7, a representative check valve is located in the valve piston 8 in the form of a check valve flapper 4. It should be understood that the mode of operation of the check valve is not constrained to a flapper-type valve and as such may be replaced by other such means as to primarily allow single directional flow. Further, one or more check valves may be associated with the valve piston 8, valve base 14, valve top 16 and/or patient connector port 2.

Returning to valve base 14, valve piston 8 is affixed thereto such that valve piston 8 is influenced by respired gas pressure coming from the patient through patient connector port 2. Valve piston 8′ operates by linear translation through a central aspect of piston guide 18 and engages upon valve top 16. Acting upon valve piston 8 is a biasing means 12, herein depicted as a representative helically wound valve spring. Biasing means 12 acts upon valve piston 8 to maintain an impingement upon valve top 16 and thereby obstructing inhaled gas from travelling from ambient/ventilation port 22 through device to patient connector port 2. Upon inhalation, either through voluntary of involuntary means, when the patient vacuum pressure exceeds the force exerted by biasing means 12, the valve piston 8 will displace and allow an increased volume of gas to vent around the perimeter of valve piston 8 through the assembly and into the patient. It should be noted that such variables as the fixed or adjustable force created by biasing means 12, the profile of valve top 16, the profile of valve piston 8, and the length of piston guide 18 can be used singularly or in combination to finitely control the minimum force, the maximum force and the flow rate through the cross-seal vent valve assembly 50.

Of particular importance in achieving improved cardiac flow by management of intrathoracic pressure is the ability of the present invention to return the intrathoracic region within anatomical norms after allowing each transient pressure vacuum window. To achieve a resetting effect of the intrathoracic pressure, and as opposed to prior art devices for such purposes, the present invention utilizes a cross-seal vent 20. In the embodiment depicted in FIG. 1-7, check valve retention 6, which is affixed to valve piston 8 for the additional purpose of retaining the position of check valve flapper 4, further comprises a centrally located fluidic pathway having a defined internal diameter forming cross seal vent 20 extending through the center of check valve retention 6. Thus a degree of fluid communication is maintained, regardless of position of valve piston 8, between patient connector port 2 and ambient/ventilation port 22, the degree of which is controlled by the cross-sectional area of cross-seal vent 20. A defining feature of cross-seal vent 20 is that the cross sectional area of cross-seal vent 20 is the smallest cumulative cross-sectional area along the fluid path(s) between patient connector port 2 and ambient/ventilation port 22 that is not interrupted by the sealing action of valve piston 8. It should be noted that cross-seal vent 20 is not constrained to a particular geometric profile, number of such vents, or flow path through valve piston 8. Similarly, the flapper valve (the combined action of check valve flapper 4 and check valve flow conduits 10) is not constrained to valve piston 8. Although a preferred embodiment is represented by inclusion of cross-seal vent 20 and the flapper valve into valve piston 8, a number of other embodiments are also equally possible. Cross-seal vent 20 is effective provided that at a minimum cross-seal vent 20 provides a limited but constant fluid communication between patient connector port 2 and the ambient environment, and more preferably with ambient/ventilator port 22. The combined action of check valve flapper 4 and check valve flow conduits 10 is also effective in a number of different embodiments provided that it provides greater means for exhaled flow from patient connector port 2 to ambient environment and more preferably to ambient/ventilator port 22 than inhalation flow in the opposite direction. The cross-seal vent 20 is integral to the performance of the valve assembly as the cross-seal vent 20 provides sufficient pressure transfer to allow the valve assembly to reset to anatomical norms after sufficient thoracic vacuum has been obtained and before subsequent chest compressions. In an exemplary embodiment of the present invention, it has been determined that optimal pressure transfer by cross-seal vent 20 for a human patient includes a vent having a cumulative effective cross-sectional diameter within the range of about 0.017 to 0.170 square inches, within 0.017 to 0.09 square inches being preferred, and within 0.06 to 0.09 square inches being most preferred. Further, the number of holes, or the resulting cross sectional area of the combined number of holes, can be altered to achieve differing performance attributes in the cross-seal vent 20 and correspondingly, cross-seal vent valve assembly 50. For example, a smaller combined cross seal vent 20 area can be used to induce a slower rate of reset in the valve assembly and a larger combined cross seal vent 20 area, realized by a bigger hole or multiple holes, can be used to induce a faster rate as the valve assembly resets through compression/relaxation cycles. It is also within the purview of the present invention that a variable cross-seal vent 20 can be employed in lieu of, or in conjunction with one or more static through-hole type vents. Such a variable vent portal can be mechanically or electronically adjusted through manual, semi-automated and fully automated processes depending upon such parameters as patient age, desired therapy, and nature of trauma. Further, one or more cross-seal vent 20 may be associated with valve piston 8, valve base 14, and/or patient connector port 2.

patient connector port 2 may include any suitable design to allow for interfacing between the valve assembly and a patient. Preferably, patient connector port 2 has a respiratory fitting having a 15 millimeter inner diameter and resulting 22 millimeter outside diameter, so as to be readily connected to a conventional sealing-type face mask, endotracheal tube or like device.

Valve piston 8 includes check valve flow conduits 10 and central piston conduit 26. The inside diameter of central conduit 26 serves as the means for the interference fit of retention boss 28 when check valve retention 6 is inserted through flapper orifice 30 and pressed into central conduit 26. Thus check valve flapper 4 is held in place on the face of valve piston 8 by check valve retention 6 over check valve flow conduits 10 such that exhalation gas is allowed to pass through check valve flow conduits 10 and around check valve flapper 4, but inhalation flow is obstructed. Thereby, inhalation gas is primarily only allowed to pass through cross-seal vent 20 until such time that the inhalation pressure is sufficient for valve piston 8 to overcome the biasing force of biasing means 12, thus valve piston 8 opens. Upon opening of valve piston 8, inhalation gas is allowed to both pass around valve piston 8 and through cross-seal vent 20. Upon exhalation, valve piston 8 returns or remains in the biased closed position but gas is allowed to travel through, in addition to cross-seal vent 20, check valve conduits 10. The geometry of check valve flow conduits 10 and the stiffness of check valve flapper 4 are such, as compared to the geometry of valve piston 8 and the force of biasing means 12, that the resistance to flow through the device during exhalation is smaller than the resistance to flow through the device during inhalation.

Check valve flapper 4 may be comprised of any suitable composition and formed by standard techniques applicable to the medical device industry. Compositions may include metallic or non-metallic substrates, with non-metallic polymers having a durometer between 10 and 110 being preferred. In the alternative, thin non-reactive polymeric materials such as silicon and Mylar in thicknesses of between 0.001 inch and 0.040 inch being preferred. Conventional injection molding or die cutting technology can be employed in the manufacture of the check valve flapper 4.

Valve base 14, valve top 16, piston guide 18, piston shaft 24 and valve piston 8 may each be comprised of the same or different suitable composition and formed by standard techniques applicable to the medical device industry. Compositions may include metallic or non-metallic substrates, with non-metallic polymers of the thermoset and/or thermoplastic types preferred. Conventional injection molding technology can be employed, and is normally one of the more preferred methods of manufacture.

According to the present invention, methods and devices for increasing cardiopulmonary circulation induced by chest compression and decompression when performing cardiopulmonary resuscitation are provided. Such methods and devices may be used in connection with any method of CPR in which intrathoracic pressures are intentionally manipulated to improve cardiopulmonary circulation. For instance, the present invention would improve standard manual CPR, “vest” CPR, CPR with a newly described Hiack Oscillator ventilatory system which operates essentially like an iron-lung-like device, interposed abdominal compression-decompression CPR, and active compression-decompression (ACD) CPR techniques. Although the present invention may improve all such techniques, the following description will refer primarily to improvements of ACD-CPR techniques in order to simplify discussion. However, the claimed methods and devices are not exclusively limited to ACD-CPR techniques.

ACD-CPR techniques are described in detail in “Active Compression-Decompression Resuscitation: A Novel Method of Cardiopulmonary Resuscitation”, Cohen et al., American Heart Journal, 1992 124(5); “Active Compression-Decompression: A New Method of Cardiopulmonary Resuscitation”, Cohen et al., The Journal of the American Medical Association, 1992, 267(21), these incorporated by reference herein in their respective entireties.

The use of a vacuum-type cup for actively compressing and decompressing a patient's chest during ACD-CPR is described in a brochure of AMBU International A/S, Copenhagen, Denmark, entitled Directions for Use of AMBU® CardioPump™, published in September 1992. The AMBU® CardioPump™ is also disclosed in European Patent Application No. 0 509 773 A1. These references are hereby incorporated by reference.

The proper performance of ACD-CPR to increase cardiopulmonary circulation is accomplished by actively compressing a patient's chest with an applicator body. Preferably, this applicator body will be a suction-type device that will adhere to the patient's chest, such as the AMBU® CardioPump™, available from AMBU International, Copenhagen, Denmark. After the compression step, the adherence of the applicator body to the patient's chest allows the patient's chest to be lifted to actively decompress the patient's chest. The result of such active compression-decompression is to increase intrathoracic pressure during the compression step, and to increase the negative intrathoracic pressure during the decompression step thus enhancing the blood-oxygenation process and enhancing cardiopulmonary circulation. ACD-CPR techniques are described in detail in Todd J. Cohen et al., Active Compression-Decompression Resuscitation: A Novel Method of Cardiopulmonary Resuscitation, American Heart Journal, Vol. 124, No. 5, pp. 1145-1150, November 1992; Todd J. Cohen et al., Active Compression-Decompression: A New Method of Cardiopulmonary Resuscitation, The Journal of the American Medical Association, Vol. 267, No. 21, Jun. 3, 1992; and J. Schultz, P. Coffeen, et al., Circulation, in press, 1994. These references are hereby incorporated by reference.

The present invention is especially useful in connection with ACD-CPR techniques. In particular, the invention improves ACD-CPR by providing methods and devices which impede air flow into or out of the patient's lungs to enhance positive or negative intrathoracic pressure during the compression or decompression of the patient's chest, thus increasing the degree and duration of a pressure differential between the thorax (including the heart and lungs) and the peripheral venous vasculature. Enhancing intrathoracic pressure with simultaneous impedance of movement of gases into or out of the airway thus enhances venous blood flow into the heart and lungs and increases cardiopulmonary circulation.

Any of the above embodiments may further include one or more CPR aiding devices into the valve assembly, wherein visual and/or aural signals are provided to the operator of the device as to both parameters relative to effectively conducting CPR (i.e. pace or rate, measure of applied force) and patient condition (i.e. pulse, return of autonomic function/respiratory response). In addition, incorporation of a valve assembly in accordance with the present invention into a self-inflating bag-type ventilator (e.g. bag valve mask “BVM” or more commonly referred to by the name AMBU-Bag”) yields a device imminently suitable for emergency CPR situations. A representative bag valve mask is described in U.S. Pat. No. 5,163,424 which is incorporated by reference herein in its entirety.

A representative CPR bag valve mask incorporating a valve assembly in accordance with the present invention allows for the use of a finitely regulated cross-seal vent valve assembly 50 can be readily employed for increasing cardiopulmonary circulation induced by chest compression and decompression. The cross-seal vent valve assembly 50 is biased against patient respiration (which may include any one of the described equivalent/alternate control means) to produce “transient pressure windows” created in the cycling of the positive and negative pressures formed in the intrathoracic region of a patient during the execution of the CPR method. These “transient pressure windows” are points where due to biasing of the valve piston 8 and cross-seal vent 20 within valve assembly 50 attached to the respiratory system of a patient, limited inhalation initiation can occur, and therefore a set negative pressure is momentarily retained with the intrathoracic region. Beneficially, the “transient pressure windows” automatically coincide with steps in the CPR method such that less force is lost in movement of air volumes and incremental pressure gains are achieved in inducing circulation of oxygenated blood in the patient. A bag valve mask, as is commonly used in the industry, can be incorporated with the current invention by connection of the mask (or endotracheal tube) to patient connector port 2 and the remaining bag valve assembly connected to ambient/ventilation port 22. When it is a suitable time in the CPR method for introducing fresh air into the patient, a self-inflating bag component of the bag valve mask is compressed, air is forced by resulting compression around valve piston 8 into the patient and the CPR method compressions can immediately resume without further delay. It should be noted that either just a self-inflating bag or a self-inflating bag with additional fluidic control valving comprised therein can be used in conjunction with the cross-seal vent valve assembly 50.

### Example

A test procedure was developed for evaluating the performance of the present invention against a no pressure management control scenario and competitive intrathoracic pressure control technologies.

An intrathoracic model was constructed by starting with two polyurethane open cell foam blocks with dimensions of 12″×12″×4″, a tensile strength of 9 psi, a density of 2.8 lbs/cubic ft, a firmness of 0.57 psi (25% deflection), and a fine cell texture type (McMasterCarr PN 8643k712). A section of foam was removed from one of the 12″×12″ faces of one of the foam blocks, hereafter referred to as the first foam block, such that a half spherical section measuring three inches in diameter by one and one half inches deep was removed from the center of the 12″×12″ face. An adjoining 2″ semi-circular conduit was then removed on the same face of the first foam block extending from the half spherical section to the mid point of one of the four edges defining the 12″×12″ face. A twelve inch length of 22 mm corrugated tubing was placed in the conduit such that one end of the 22 mm corrugated tubing was positioned in the center of the removed half spherical section and that the other end was allowed to remain free outside the perimeter of the first foam block (extending roughly 6″ there from).

A section of foam was removed from a 12″×12″ face of the remaining unmodified foam block, hereafter referred to as the second foam block, having an elliptical profile traced on said face with a primary axis of 8 inches and a secondary axis of 2 inches and a linear depth of 3 inches. Said elliptical profile was positioned upon said 12″×12″ face of second foam block such that the end of the primary axis was positioned at the edge of 12″×12″ face, 2 inches from one of the corners of 12″×12″ face with said primary axis aligned and parallel with the immediate adjacent edge of said 12″×12″ face. An additional amount of foam was removed at the point at which the primary elliptical axis made contact with an edge of the 12″×12″ face whereby a 1″×1″ conduit was created into adjoining 12″×4″ face.

A 0.5 liter hyperinflation bag (a bag with little or no elastic return for volumes up to 0.5 liters, and elastic return for volumes greater than 0.5 liters) was obtained having a stiff open end with a 22 mm ID (such as found in Mercury Medical product number 10-55800). Two 15 inch lengths of clear vinyl tubing with an OD of 7/16″ and an ID of 5/16″ were inserted into the open end of the hyperinflation bag such that one tube's end was positioned 2 inches into the hyperinflation bag and the other tube's end was inserted 6.5 inches into the hyper inflation bag. A hot-melt adhesive was used to durably and sealably affix the two vinyl tubes into the ID of the hyperinflation bag such that the only fluid communication between the ambient environment and the inside of the hyperinflation bag was by way of the two positioned vinyl tubes. A 0.030″ thick layer of liquid latex rubber was than coated over a region of one inch about the joint formed by the vinyl tubing and the immediately adjacent stiff open end of the hyperinflation bag. The coat of liquid latex rubber was allowed to dry and then recoated in exactly the same manner for a total of 6 layers.

Upon drying overnight the hyperinflation bag assembly described above was placed in the elliptical profile cavity created in the second foam block such that the hyperinflation bag was centered in the elliptical profile, the stiff open end being centered in the 1″ conduit connected to the 12″×4″ face. The elliptical cavity, containing the hyperinflation bag, was then covered with six 8″ lengths of 2″ wide high strength cloth adhesive tape such that: each edge overlapped an adjacent piece of tape; all lengths of tape were perpendicular to the primary axis of the elliptical profile; and, that each length of tape wrapped to the nearest adjacent face by at least two. Similarly the interstitial opening between the stiff open end of the hyperinflation bag and the 1″ conduit in the second foam block was covered in such that the entire interstitial opening was protected by a cloth tape multi-laminate layer.

The first foam block was then placed upon the horizontal surface of the second foam block such that the 12″×12″ face containing the half spherical space and the connecting conduit wherein in alignment such that the elliptical profile cavity was facing directly up and in such a manner that all four 4″×12″ sides of the second foam block were aligned above and coincident with the corresponding 4″×12″ sides of the first foam block. Three 60″ lengths of 2″ wide adhesive cloth taper were then wrapped circumferentially around the mating edge of the two foam blocks such that a four inch tall horizontal retentive wrap held the two foam blocks together along the entire exposed mating edges/perimeter of the two blocks.

The top and all sides of the entire assembly described immediately above were coated with approximately 0.020″-0.040″ of liquid latex rubber with particular attention and additional liquid latex rubber added to the geometric position where the 22 mm corrugated tubing and the two vinyl tubes protruded from the assembly. Liquid latex rubber was coated an additional one to two inches down the length of each of the three tubes from the point at which each protruded from the face of the foam block assembly. The assembly was allowed to sit and dry. Once dry, the assembly was turned over such that the other 12″×12″ face was facing upward and the coating process was repeated. The cycle was repeated until the entire assembly had approximately a 0.125″ thick latex shell around the entire foam assembly.

Two pieces of ¼″ thick clear acrylic pieces measuring 3″×9″ were glued together using a solvent bonding technique such that the two pieces were butt joined along their respective 9″ edges and the 3″ axis's were perpendicular. A third piece of ¼″ thick of clear acrylic measuring 1″×2″ was than similarly butt joined at the end and inside corner such that the resulting acrylic assembly made an inside corner with the 1″ edge of the third piece of acrylic adjoined to the 3″ edge of the first piece of acrylic and the 2″ edge of the third piece of acrylic adjoined to the 3″ adjacent edge of the second piece of acrylic. The acrylic assembly was than placed on the foam block assembly such that the corner immediately adjacent to the elliptical profile was fitted into the inside corner created by the acrylic assembly with such orientation that third piece of acrylic was proximal to the exit point of the vinyl tubes.

One elastic cord having a diameter of ¼″ was then wrapped around the resulting foam, latex, and acrylic assembly such that each 8″×12″ vertical face had approximately 4 to 6 wraps with each end of the elastic cord tied off to a 2 inch diameter steel ring coincident with top face of resulting assembly. Said elastic cord was repositioned and tightened until an added gas volume of 600 ml to free end of said 22 mm corrugated tubing resulted in internal pressure of 15 cm of water column pressure.

Simple 22 mm diameter flapper valves were fitted to the free ends of said 2 lengths of vinyl tubing protruding from the foam assembly such that the first flapper valve only allowed liquid to flow into said hyperinflation bag and the second flapper valve only allowed liquid to flow out of said hyperinflation bag. Additional vinyl tubing having an ID of 5/16″ and an OD of 7/16″ was attached to free ends of said flapper valves. Free end of vinyl tubing connected to the free end of first flapper valve was caused to be in fluid communication with a free standing 4 liter reservoir of liquid water at the same elevation as the foam assembly. Free end of vinyl tubing connected to second flapper valve was positioned in an open and empty jar so as to capture fluid caused to pass through simulated heart (said hyperinflation bag) during chest compressions on simulated thoracic cavity (said foam assembly) with various devices connected to simulated trachea (said 22 mm corrugated tubing).

Utilizing the previously described thoracic model, four different conditions were tested: negative control sample without pressure management means, a commercially available device in accordance with the '394 US patent to Lurie et al., a representative device in accordance with a first embodiment of the present invention with varying cross-seal vent diameters. The intrathoracic model was prepared for each test condition by flushing through the check valves connected to the hyperinflation bag cardiac sub-assembly for 15 minutes to remove any trapped air. Water was used to represent a blood substitute and to establish a means for determine liquid flow resulting from ten (10) consecutive chest compressions executed using each test condition. The results are presented in Table 1 below. Chest compressions were induced per U.S. National standards of one hand placed over another and full weight compression was realized at the center point of the 12″×12″ face that incorporated the elliptical profile and the simulated heart. During simulated CPR the model was placed at a height of about 40 inches above the ground. The adult male performing the CPR was of sufficient size to produce significant results having a height of 72″, a weight of 200 lbs, a shoulder size of 44 inches, and a body fat content of less than 20%.

TABLE 1
“Cardiac” Flow per Chest Compression

| | | Standard |
|---|---|---|
| | Mean (ml | Deviation (ml |
| Test Condition | flow/compression) | flow/compression) |
| No Pressure | 0.33 | 0.10 |
| Control Device | | |
| U.S. Pat. No. ′394 | 0.59 | 0.08 |
| Device | | |
| Cross-Seal Vent | 0.60 | 0.12 |
| Diameter = 0.017″ | | |
| Cross-Seal Vent | 0.70 | 0.08 |
| Diameter = 0.062″ | | |
| Cross-Seal Vent | 0.76 | 0.11 |
| Diameter = 0.071″ | | |
| Cross-Seal Vent | 0.63 | 0.10 |
| Diameter = 0.088″ | | |
| Cross-Seal Vent | 0.54 | 0.10 |
| Diameter = 0.150″ | | |

As can be seen in Table 1, an intrathoracic pressure control means having at least one cross-seal vent valve in association with the respiratory pathway of a simulated patient receiving CPR improves flow rate by at least twice as much over a no pressure control condition and by at least 25% over a continuous vacuum methodology as represented by a device in accordance with U.S. Pat. No. '394 to Lurie et al, incorporated previously by reference.

From the foregoing, it will be observed that numerous modifications and variations can be affected without departing from the true spirit and scope of the novel concept of the present invention. It is to be understood that no limitation with respect to the specific embodiments illustrated herein is intended or should be inferred. The disclosure is intended to cover, by the appended claims, all such modifications as fall within the scope of the claims.

What is claimed:

1. A device for finitely regulating intrathoracic pressure comprising;
a. a patient connector;
b. a valve piston assembly;
c. a cross-seal vent;
wherein said valve piston assembly is triggered to open and to reset to close in response to pressure changes;
wherein said cross-seal vent allows for venting of pressure from within said valve piston assembly;
wherein a patient connected to said device experiences a change in intrathoracic pressure resulting from the triggering and resetting of said device.

2. A device as in claim 1, wherein said cross-seal vent is associated with said patient connector.

3. A device as in claim 1, wherein said cross-seal vent is associated with said valve piston assembly.

4. A device as in claim 1, wherein said pressure is formed by a patient connected to said device.

5. A device as in claim 1, wherein said change in intrathoracic pressure forms transient pressure windows.

6. A method for enhancing the performance of cardiopulmonary resuscitation comprising;
a. connecting a patient's respiratory pathway to a device capable of finitely regulating intrathoracic pressure;
b. performing cardiopulmonary resuscitation;
wherein said device capable of finitely regulating intrathoracic pressure comprises a valve assembly.

7. A method as in claim 6, wherein said device capable of finitely regulating intrathoracic pressure further comprises a cross-seal vent.

8. A device as in claim 7, wherein said cross-seal vent is associated with a patient connector.

9. A device as in claim 7, wherein said cross-seal vent is associated with said valve assembly.

10. A method as in claim 6, wherein said device is used to treat patients having compromised pulmonary performance.

11. A method as in claim 6, wherein said device is used to treat patients so as to obtain enhanced pulmonary performance.

12. A method for increasing blood flow to the thorax by augmenting negative intrathoracic pressures for a finite period of time, said method comprising the steps of:
a. Interfacing at least one valve assembly to a patient's airway; and
b. Manipulating a body structure of a patient to increase the magnitude and duration of the patient's negative intrathoracic pressure, wherein during said manipulation of a body structure of a patient said valve assembly obstructs respiratory gases entering the lungs until a negative intrathoracic pressure level in the range from about 0 cm H₂O to −30 cm H₂O is exceeded at which time said valve assembly reduces said obstruction, said valve assembly assisting in increasing the magnitude and duration of negative intrathoracic pressure thereby enhancing the amount of blood flow into the heart and lungs; and
c. A reduction of patient's negative intrathoracic pressure within 1 second of said manipulation of a body structure of a patient.

13. The method of claim 12, further comprising the restricted flow of gas from the ambient environment to the patient's airway during a period of negative intrathoracic pressure.

14. The method of claim 12, wherein the manipulation step comprises performing chest compression and chest decompression, and wherein the chest decompression step comprises allowing the patient's chest to expand in response to the chest's resilience.

15. The method of claim 12, wherein the manipulation step comprises lifting or actively expanding the patient's chest to expand the thorax

16. The method of claim 12, wherein the magnitude of the reduction of patient's negative intrathoracic pressure within 1 second of said manipulation of a body structure of a patient is at least fifty percent of the peak negative intrathoracic pressure realized during said manipulation of a body structure of a patient.
